(19) United States
(12) Patent Application Publication
(10) Pub. No.: US 2005/0250804 A1
(43) Pub. Date: Nov. 10, 2005

(54) Pharmaceutical gel formulations

(75) Inventors: Muthaiyyan Esakki Kannan, Kopar Khairane (IN); Suma Prakasan Nair, Dombivli (IN); Anandi Krishnan, Vashi (IN)

Correspondence Address: DILWORTH & BARRESE, LLP, 333 EARLE OVINGTON BLVD., UNIONDALE, NY, 11553, US

(73) Assignee: Glenmark Pharmaceuticals Limited, Mumbai (IN)

(21) Appl. No.: 11/124,345

(22) Filed: May 6, 2005

Related U.S. Application Data
(60) Provisional application No. 60/568,529, filed on May 6, 2004.

(51) Int. Cl. A61K 31/4745

(52) U.S. Cl. 514/291

(57) ABSTRACT

A pharmaceutical gel composition is provided comprising (a) a therapeutically effective amount of one or more active pharmaceutical ingredients comprising one or more macrolide related immunosuppressants or pharmaceutically acceptable salts or esters thereof; (b) one or more gel forming agents; and (c) an effective amount of one or more skin penetration enhancers capable of percutaneous delivery of the macrolide related immunosuppressant through the skin. Also provided is a process for its preparation and methods for delivering a macrolide related immunosuppressant through the skin of a mammal in order to treat conditions situated on and beneath the skin.

### CROSS-REFERENCE TO RELATED APPLICATIONS

This application claims the benefit under 35 U.S.C. §119 to Provisional Application No. 60/568,529, filed May 6, 2004 and entitled “TOPICAL MACROLIDE PHARMACEUTICAL GEL FORMULATIONS”, the contents of which are incorporated by reference herein.

### BACKGROUND OF THE INVENTION

1. Technical Field

The present invention relates generally to pharmaceutical gel formulations.

2. Description of the Related Art

The administration of drugs and other biological materials to the bloodstream via a transdermal route or to the localized site of action has received much attention in recent years. The skin of an average adult covers generally more than two square meters of surface area and receives about one third of all blood circulating through the body. It is elastic, rugged, and generally self generating. The skin consists of three layers: the stratum corneum, the epidermis, and the dermis.

The stratum corneum represents the rate limiting step in diffusion of chemicals through the skin. The stratum corneum is composed of dead, keratinized, metabolically inactive cells, which are closely packed together, and consists of an amorphous matrix of mainly lipoid and non fibrous protein within which keratin filaments are distributed. The cells of the stratum corneum generally contain about 20% water, while the cells below, in the stratum germinivatum contain about 70% water. The stratum corneum does not become hydrated readily. Thus, transdermal permeation is primarily controlled by diffusion through the stratum corneum.

Due to availability of large surface area, easy accessibility, application dynamics and the noninvasive nature of the therapy, topical administration of drugs has long been considered a promising route of drug delivery whether the bioavailability desired is systemic, dermal, regional or localized. The topical mode of drug delivery provides many advantages over customarily used routes of administration. First, it bypasses the portal circulation and thereby the hepatic first pass metabolism. Second, topical delivery avoids the problems of variable systemic absorption and metabolism. Third, it potentially reduces gastrointestinal irritation associated with oral administration. Further, it avoids the risks and patient noncompliance associated with parenteral treatment.

The topical delivery route offers continuity of drug administration, permits use of therapeutic agents with short biological half lives, provides treatment of cutaneous manifestations of diseases usually treated systemically delivers medication directly into the systemic circulation and fosters ease of use and total patient compliance.

Gels are semisolid systems consisting of dispersions of small or large molecules in an aqueous liquid vehicle rendered jelly-like through the addition of a gelling agent. In addition to the gelling agent and water, gels may be formulated to contain an active pharmaceutical ingredient (API), co-solvents, anti-microbial preservatives, stabilizers, and other excipients. Medicated gels may be prepared for administration by various routes including topically to the skin, to the eye, nasally, vaginally, and rectally.

Several pharmaceutical compositions are described in literature for topical application of macrolide related immunosuppressant drugs which are known to exhibit efficacy in the treatment of autoimmune related skin conditions.

One example is Protopic® (tacrolimus) ointment which contains tacrolimus, a macrolide immunosuppressant produced by Streptomyces tsukubaensis. It is typically utilized for topical dermatologic use only. Chemically, tacrolimus is designated as [3S-[3R*[E(1S*,3S*,4S*)],4S*,5R*,8S*,9E, 12R*,14R*,15S*,16R*,18S*,19S*,26aR*]]-5,6,8,11,12,13,14,15,16,17,18,19,24,25,26,26a-hexadecahydro-5,19-dihydroxy-3-[2-(4-hydroxy-3 -methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone, monohydrate as shown in FIG. I: 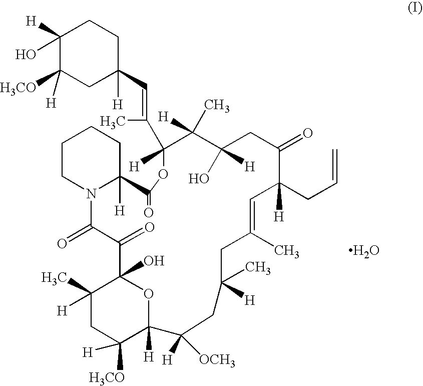
Tacrolimus has an empirical formula of C₄₄H₆₉NO₁₂.H₂O and a formula weight of 822.03. It has been demonstrated that tacrolimus inhibits T lymphocyte activation by first binding to an intracellular protein, FKBP-12. A complex of tacrolimus-FKBP-12, calcium, calmodulin, and calcineurin is then formed and the phosphatase activity of calcineurin is inhibited. This effect has been shown to prevent the dephosphorylation and translocation of nuclear factor of activated T-cells (NF-AT), a nuclear component thought to initiate gene transcription for the formation of lymphokines (such as interleukin-2, gamma interferon). Tacrolimus also inhibits the transcription for genes which encode IL-3, IL-4, IL-5, GM-CSF, and TNF-α, all of which are involved in the early stages of T-cell activation. Additionally, tacrolimus has been shown to inhibit the release of pre-formed mediators from skin mast cells and basophils, and to down regulate the expression of FceRI on Langerhans cells.

Tacrolimus is indicated for short-term and intermittent long-term therapy in the treatment of patients with moderate to severe atopic dermatitis in whom the use of alternative, conventional therapies are deemed inadvisable because of potential risks, or in the treatment of patients who are not adequately responsive to or are intolerant of alternative, conventional therapies. See, e.g., Physician's Desk Reference, “Protopic,” 58th Edition, p. 1327-1330 (2003).

Another example is Elidel® (pimecrolimus) Cream 1% which contains the compound pimecrolimus, the 33-epi-chloro-derivative of the macrolactam ascomycin. Chemically, pimecrolimus is (1R,9S,12S,13R,14S,17R,18E,21 S,23S,24R,25S,27R)-12-[(1E)-2-{(1R,3R,4S)-4-chloro-3-methoxycyclohexyl}-1-methylvinyl]-17-ethyl-1,14-dihydroxy-23,25-dimethoxy-13,19,21,27-tetramethyl-11,28-dioxa-4-aza-tricyclo[22.3.1.04,9]octacos-18-ene-2,3,10,16-tetraone as shown in FIG. II: 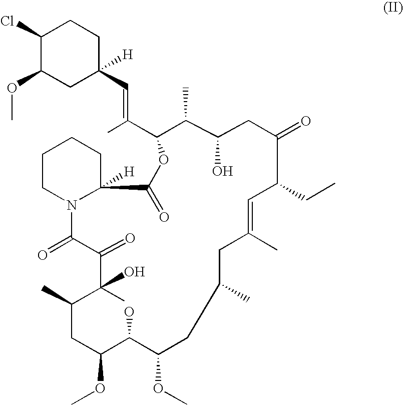
Pimecrolimus has the empirical formula C₄₃H₆₈ClNO₁₁ and a molecular weight of 810.47. Pimecrolimus has been demonstrated to bind with high affinity to macrophilin-12 (FKBP-12) and inhibit the calcium-dependent phosphatase, calcineurin. As a consequence, it inhibits T-cell activation by blocking the transcription of early cytokines. In particular, pimecrolimus inhibits at nanomolar concentrations Interleukin-2 and interferon gamma (Th1-type) and Interleukin-4 and Interleukin-10 (Th2-type) cytokine synthesis in human T cells. In addition, pimecrolimus prevents the release of inflammatory cytokines and mediators from mast cells in vitro after stimulation by antigen/IgE. Pimecrolimus is indicated for short-term and intermittent long-term therapy in the treatment of mild to moderate atopic dermatitis. See, e.g., Physician's Desk Reference, “Elidel,” 58th Edition, p. 2250-2252 (2003).

Yet another example is Rapamune® (sirolimus) which is an immunosuppressive agent. Sirolimus is a macrocyclic lactone produced by Streptomyces hygroscopicus. The chemical name of sirolimus (also known as rapamycin) is (3 S,6R,7E,9R,10R,12R,14S,15SE,17E,19E,21S,23S,26R,27R,4aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]oxaazacyclohentriacontine-1,5,11,28,29(4H,6H,31H)-pentone as shown in FIG. III: 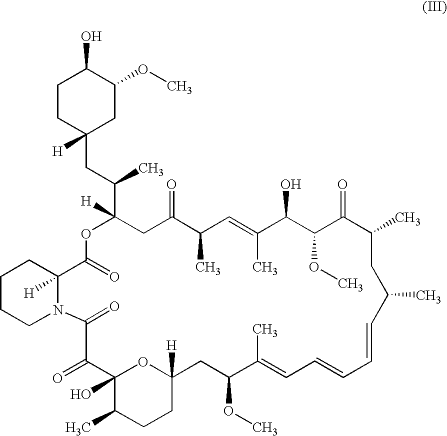
Sirolimus has a molecular formula of C₅₁H₇₉NO₁₃ and its molecular weight is 914.2. Sirolimus inhibits T lymphocyte activation and proliferation that occurs in response to antigenic and cytokine (Interleukin [IL]-2, IL-4, and IL-15) stimulation by a mechanism that is distinct from that of other immunosuppressants. Sirolimus also inhibits antibody production. In cells, sirolimus binds to the immunophilin, FK Binding Protein-12 (FKBP-12), to generate an immunosuppressive complex. The sirolimus:FKBP-12 complex has no effect on calcineurin activity. This complex binds to and inhibits the activation of the mammalian Target Of Rapamycin (mTOR), a key regulatory kinase. This inhibition suppresses cytokine-driven T-cell proliferation, inhibiting the progression from the G1 to the S phase of the cell cycle. Sirolimus is indicated for the prophylaxis of organ rejection in patients receiving renal transplants. See, e.g., Physician 's Desk Reference, “Rapamune,” 58th Edition, p. 3483-3490 (2003).

### SUMMARY OF THE INVENTION

In accordance with one embodiment of the present invention, a pharmaceutical gel composition is provided comprising (a) a therapeutically effective amount of one or more active pharmaceutical ingredients comprising one or more macrolide related immunosuppressants or pharmaceutically acceptable salts or esters thereof; (b) one or more gel forming agents; and (c) an effective amount of one or more skin penetration enhancers capable of percutaneous delivery of the macrolide related immunosuppressant through the skin.

In accordance with a second embodiment of the present invention, a process for preparing a pharmaceutical gel composition is provided comprising:
(a) dissolving a therapeutically effective amount of one or more active pharmaceutical ingredients comprising one or more macrolide related immunosuppressants or pharmaceutically acceptable salts or esters thereof in one or more penetration enhancers to form a first solution;
(b) adding one or more solvents to the first solution;
(c) mixing one or more gel forming agents in water to form a second solution; and
(d) mixing the second solution with the product of step (b).

In accordance with a third embodiment of the present invention, a method for delivering one or more macrolide related immunosuppressants or pharmaceutically acceptable salts or esters thereof through the skin in order to treat conditions situated on and beneath the skin is provided, the method comprising the step of topically administering an effective amount of a pharmaceutical gel composition comprising (a) a therapeutically effective amount of one or more active pharmaceutical ingredients comprising one or more macrolide related immunosuppressants or pharmaceutically acceptable salts or esters thereof; (b) one or more gel forming agents; and (c) an effective amount of one or more skin penetration enhancers capable of percutaneous delivery of the macrolide related immunosuppressant through the skin.

### DEFINITIONS

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

By “pharmaceutically acceptable” is meant those salts and esters which are, within the scope of sound medical judgment, suitable for use in contact with the tissues of humans and lower animals without undue toxicity, irritation, allergic response and the like, commensurate with a reasonable benefit/risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarare, succinate, tartrate, ascorbate, glucoheptonate, lactobionate, lauryl sulphate salts and the like. Representative alkali or alkaline earth metal salts include the sodium, calcium, potassium and magnesium salts, and the like.

The term “subject” or “a patient” or “a host” as used herein refers to mammalian animals, preferably human.

As used herein the term “antioxidant” is intended to mean an agent who inhibits oxidation and is thus used to prevent the deterioration of preparations by the oxidative process. Such compounds include, by way of example and without limitation, ascorbic acid, ascorbic palmitate, Vitamin E, butylated hydroxyanisole, butylated hydroxytoluene, hypophosphorous acid, monothioglycerol, propyl gallate, sodium ascorbate, sodium bisulfite, sodium formaldehyde sulfoxylate, sodium metalbisulfite and other such materials known to those of ordinary skill in the art.

As used herein, the term “buffering agent” is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ f127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### DETAILED DESCRIPTION OF THE PREFERRED EMBODIMENTS

One aspect of the present invention provides a pharmaceutical gel composition comprising (a) a therapeutically effective amount of one or more active pharmaceutical ingredients comprising one or more macrolide related immunosuppressants or pharmaceutically acceptable salts or esters thereof; (b) one or more gel forming agents; and (c) an effective amount of one or more skin penetration enhancers capable of percutaneous delivery of the macrolide related immunosuppressant through the skin. The terms “active”, “drug”, and “active pharmaceutical ingredient” are used interchangeable herein. The macrolide related immunosuppressants for use herein can be any known macrolide immunosuppressants and includes, but are not limited to, tacrolimus, pimecrolimus, sirolimus, acsomycin, everolimus, pharmaceutically acceptable salts thereof and the like and mixtures thereof.

As will be readily be understood by those skilled in the field of pharmaceutical formulation, gels are semisolid, suspension-type systems. Gel forming agents for use herein can be any gelling agent typically used in the pharmaceutical art for topical semi solid gel dosage forms. As used herein, the term “gelling agent” is intended to mean a compound used to render a liquid vehicle into a jelly-like vehicle. Exemplary gelling agents include, by way of example and without limitation, synthetic macromolecules, cellulose derivatives (e.g. carboxymethylcellulose and hydroxypropylmethyl-cellulose) and natural gums (e.g. tragacanth). The synthetic macromolecules include carbomers (e.g. Carbomer 910, 934, 934P, 940, 941, and 1342), which are high molecular weight water-soluble polymers of acrylic acid cross-linked with allyl ethers of sucrose and/or pentaerythritol. Carbomers have different viscosities depending on their polymeric composition. Gelling agents of the present invention may be selected from any of synthetic or semi-synthetic polymeric materials, polyacrylate copolymers, cellulose derivatives and polymethyl vinyl ether/maleic anhydride copolymers. Various grades of Carbopol such as, for example, Carbopol 934, 940, 941, 974, 980, 981, 1342, 5984, ETD2020, ETD 2050, and Ultrez 10 (available from Noveon of Cleveland, Ohio) can be used in the present invention. The present invention preferably includes Carbopol 980 as a gelling agent. A Carbopol is a carbomer. Generally, carbomers are synthetic high molecular weight polymer of acrylic acid that are cross linked with either allylsucrose or allylethers of pentaerythritol.

The gelation mechanism depends on neutralization of the carboxylic acid moiety to form a soluble salt. The polymer is hydrophilic and produces sparkling clear gels when neutralized. Carbomer gels possess good thermal stability in that gel viscosity and yield value are essentially unaffected by temperature. As a topical product, carbomer gels possess optimum rheological properties. The inherent pseudo plastic flow permits immediate recovery of viscosity when shear is terminated and the high yield value and quick break make it ideal for dispensing. In the present pharmaceutical formulations, carbomer gels are used as a suspending or viscosity increasing agent. Aqueous solution of Carbopol is acidic in nature due to the presence of free carboxylic acid residues. Neutralization of this solution crosslinks and gelatinizes the polymer to form a viscous integral structure of desired viscosity. The amount of gelling agents varies widely and will ordinarily range from about 0.1% to about 10% w/w.

“Penetration enhancement” or “permeation enhancement” as used herein relates to an increase in the permeability of the skin or mucosal tissue to the selected pharmacologically active agent, which in turn gives rise to an increase in the rate at which the drug permeates into and/or through the skin or mucosal tissue. Accordingly, the skin penetration enhancers herein are employed to improve the permeability of an active pharmaceutical ingredient through the skin. Exemplary penetration enhancers include, by way of example and without limitation, volatile organic solvents (e.g. alcohols such as ethanol), nonvolatile organic solvents (e.g. amides such as pyrrolidones; polyol ethers such as glycol ethers; polyols such as glycols; and derivatives thereof) and the like and mixtures thereof.

Suitable volatile organic solvents include, but are not limited to, aliphatic, cycloaliphatic and/or aromatic-aliphatic alcohols, each of which is monohydric or polyhydric, alcohol/water mixtures, saturated and/or unsaturated fatty alcohols which each contain from about 8 to about 18 carbon atoms, saturated and/or unsaturated fatty acids which each from about 8 to about 18 carbon atoms and/or esters thereof and the like and mixtures thereof. Useful alcohols are those having from 1 to about 20 carbon atoms, e.g., ethanol, isopropyl alcohol, etc. Topical ethanol solutions can also be used as a penetration enhancer at a concentration ranging from about 1% v/v to about 90% v/v. See, e.g., A. Kibbe, Handbook of Pharmaceutical Excipients, 3rd Ed. (2000). The amounts of ethanol may vary greatly, even outside the ranges given by the Handbook of Pharmaceutical Excipients. In the present invention, an alcohol can be used as co-solvent cum penetration enhancer for the API. While not being bound to any particular theory, there are two theories supporting the use of alcohol as skin penetration enhancer. First, the alcohol evaporates fast and concentrates the drug in the residual formulation that remains on the skin. It is believed that some thermodynamic activity will drive the drug into the stratum corneum. Second, the alcohol alters the physical integrity of the stratum corneum barrier resulting in an increase in the ability of the drug to penetrate the skin. Commercially, denatured alcohol such as SDA 40 is often used in place of Alcohol USP (ethanol), and it may be used here also. A preferred alcohol is ethanol.

Suitable amides for use as nonvolatile organic solvents include, but are not limited to, N,N-dimethyl acetamide (DMA) N,N-diethyl toluamide, N,N-dimethyl formamide, N,N-dimethyl octamide, N,N-dimethyl decamide, and the like; pyrrolidone derivatives such as N-alkylpyrrolidones (e.g., N-methyl-2-pyrrolidones), vinyl pyrrolidone, 2-pyrrolidone, 2-pyrrolidone-5-carboxylic acid, N-(2-hydroxyethyl)-2-pyrrolidone or fatty acid esters thereof, 1-lauryl-4-methoxycarbonyl-2-pyrrolidone, N-tallowalkylpyrrolidones, and the like and mixtures thereof. The present invention preferably employs N-methyl-2-pyrrolidone as a nonvolatile type of penetration enhancer. N-methyl-2-pyrrolidone is commercially available as Pharmasolve® (available from ISP Corp. of Wayne, N.J.), a safe and compatible material. Generally, Pharmasolve® increases the water solubility of insoluble drugs and can therefore develop insoluble drugs into topical products.

Solubility enhancement can be attributed to three parameters: nonpolar molecular dispersion, polar type chemical bonding and hydrogen bonding. N-methyl-2-pyrrolidone can undergo a large number of chemical reactions at various positions on the pyrrolidone ring. The enhanced solubility can be attributed to a complexing action with the nitrogen and carbonyl reactive centers of the molecule. A preferred N-methyl-2-pyrrolidone is Pharmasolve® which is a drug solubilizer and therefore enhances the bioavailability of topical formulations. It can increase the solubility of many drugs that are not water-soluble thereby enhancing their physico-chemical stability and bioavailability.

Pharmasolve's® favorable safety profile makes it a solubilizer of choice for use in pharmaceutical formulations. Furthermore, in contrast to other drug solubilizing agents, Pharmasolve® has a favorable toxicity profile, making it a suitable candidate for use in a variety of topical dosage forms. Pharmasolve® is practically nonirritating to rabbit skin. (PII=0.5). According to the manufacturer's brochure, when Pharmasolve® was analyzed in a repeated insult patch test using 50 human subjects and a total of 15 applications, N-methyl-2-pyrrolidone was neither a primary dermal irritant nor a sensitizer.

Polyol ethers for use herein can be C₂-C₃₀ polyol ethers containing from 2 to about 10 hydroxyl groups. Representative of the polyol ethers are glycol ethers which include, by way of example and without limitation, ethylene glycol monomethyl ether, ethylene glycol monoethyl ether, ethylene glycol monobutyl ether, ethylene glycol monopropyl ether, ethylene glycol monophenyl ether, ethylene glycol monohexyl ether, diethylene glycol monoethyl ether, diethylene glycol monomethyl ether, triethylene glycol monomethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethyl glycol monohexyl ether, ethylene glycol phenyl ether, polyethylene glycol, polyethylene glycol dodecyl ether, diethylene glycol monoethyl ether, polyethylene glycol-8-glyceryl caprylate and the like and mixtures thereof.

Diethylene glycol monoethyl ether, commercially available as Transcutol® (available from Gattefosse of St Priest Mi-Plaine, France), is a preferred polyol ether. Transcutol® can solubilize hydrophobic materials. The increased drug flux across the stratum corneum is explained by the diffusion of Transcutol® into it, thereby changing the ability of the drug to penetrate the intercellular space. It is believed that the primary role of Transcutol® is the modification of the thermodynamic activity of the drug. By direct action on its solubility, Transcutol® favors the passage of larger quantities of the drug into the stratum corneum allowing a greater solubilization in the aqueous domains of the tissue. Currently, there are various clinical studies conducted in the U.S. using Transcutol® in topical products (Phase I, II and III clinical trials).

Polyols for use herein can be C₂-C₃₀ polyols containing from 2 to about 10 hydroxyl groups. Suitable polyols according to the present invention include, but are not limited to, ethylene glycol, propylene glycol, butylene glycol, hexylene glycol, propylene glycol monocaprylate and mixtures thereof. Propylene glycol is widely used as a solvent or a co-solvent in topicals ranging from about 1% to about 90%. It is also used as a humectant in an amount of about 15%. See, e.g., A. Kibbe, Handbook of Pharmaceutical Excipients, 3rd Ed. (2000). In hydro alcoholic gel formulations, alcohol evaporates, propylene glycol and water forms a saturated solution of drug, which enhances flux of drug across the skin.

In one embodiment, the skin penetration enhancers for use in the compositions of the present invention will be the amides such as the foregoing pyrrolidones and will include one or more solvents that are pharmaceutically acceptable for application to skin or exposed tissue of a non-human. Suitable solvents include, but are not limited to, C₁-C₄ alcohols, C₁-C₄ alkylene glycols, C₁-C₄ polyalcohols, C₁-C₄ polyalkylene glycols, sorbates, polysorbates, benzyl alcohol, triglycerides, and water. Specific examples of suitable components for the solvent mixture include propylene glycol, glycerin, ethanol, isopropyl alcohol and the like. Specifically, propylene glycol, glycerin, isopropyl alcohol, ethanol, and the like, are recognized in the art as safe for topical application to non-human skin and/or exposed tissue.

For example, propylene glycol can serve as a moisturizer and can produce a pleasant emollient feel when applied to the skin. Furthermore, propylene glycol also has the added advantage of being a mild germicide. However, in excessive concentrations the germicidal properties can potentially irritate sensitive skin.

In one example of a suitable embodiment, the solvent mixture includes an alcohol such as ethanol in an amount of about 1% to about 90% w/w and preferably from about 5% to about 50% w/w and a glycol such as propylene glycol in an amount of about 1% to about 90% w/w and preferably from about 5% to about 50% w/w.

The active pharmaceutical ingredient(s) will be present in an amount effective to prevent, treat or aid in the healing of a human skin or tissue disorder. The precise amount of active pharmaceutical ingredient is dependent upon both the disorder and the human being treated and optimization would therefore involve only routine experimentation. Generally, the amount of the active pharmaceutical ingredient present in the topical pharmaceutical compositions of the present invention can range from about 0.01% to about 5 %w/w and preferably from about 0.02% to about 2% w/w. The penetration enhancer will ordinarily be present in an amount sufficient to enhance the penetration of the macrolide related immunosuppressant into the skin. The specific amount varies necessarily according to the desired release rate and specific form of the macrolide related immunosuppressant used. Generally, this amount is in the range of about 0.01% to about 30% w/w and preferably from about 1% to about 10% w/w to be administered to a patient.

The compositions of the present invention may further include a neutralizing agent to adjust the pH of the composition in the range of about 3.0 to about 6.5. Useful neutralizing agents include, but are not limited to, organic basic compounds, inorganic basic compounds and the like and mixtures thereof. Examples of organic basic salts of the present invention include C₁-C₂₀ alkanolamines, such as methanolamine, ethanolamine, propanolamine, butanolamine, dimethanloamine, diethanolamine, dipropanolamine, dibutanolamine, diisopropanolamine, tributanolamine, aminomethylpropanol, N-methyl-glucamine, tetrahydroxypropyl ethylene diamine and the like, C₁-C₂₀ alkylamines such as methylamine, ethylamine, propylamine, butylamine, diethylamine, dipropylamine, isopropylamine and the like and mixture thereof. Examples of inorganic basic salts of the present invention include ammonium hydroxide, alkali metal salts, alkaline earth metal salts such as magnesium oxide, magnesium hydroxide, calcium hydroxide, sodium hydroxide, potassium hydroxide, lithium hydroxide, aluminum hydroxide, potassium carbonate, sodium bicarbonate and the like.

If desired, the topical pharmaceutical compositions of the present invention can contain additional active pharmaceutical ingredients other than the aforementioned macrolide related immunosuppressants depending on the particular condition being treated. The topical pharmaceutical compositions of the present invention can also include one or more pharmaceutically acceptable excipients that are typically used in the art for locally applied semisolid gel dosage forms. Suitable pharmaceutically acceptable excipients include, but are not limited to, antioxidants, buffering agents, binders, wetting agents, microbial preservatives, stabilizers and the like and mixtures thereof.

The topical pharmaceutical composition herein can be formulated into any suitable formulation such as, for example, immediate release formulation, controlled release formulation, fast melt formulation, delayed release formulation, extended release formulation, mixed release formulations such as immediate release and controlled release formulations and the like.

The pharmaceutical gel compositions of the present invention can be obtained by (a) dissolving a therapeutically effective amount of one or more active pharmaceutical ingredients comprising one or more macrolide related immunosuppressants or pharmaceutically acceptable salts or esters thereof in one or more penetration enhancers to form a first solution; (b) adding one or more solvents to the first solution; (c) mixing one or more gel forming agents in water to form a second solution; (d) mixing the second solution with the product of step (b); and, optionally, (e) mixing one or more neutralizing agents with the solution.

Another embodiment of the present invention is a method for delivering a macrolide related immunosuppressant through the skin of a mammal in order to treat conditions situated on and beneath the skin including at least the step of topically administering to the skin of the mammal an effective amount of a pharmaceutical gel composition comprising (a) a therapeutically effective amount of one or more active pharmaceutical ingredients comprising one or more macrolide related immunosuppressants or pharmaceutically acceptable salts or esters thereof; (b) one or more gel forming agents; and (c) an effective amount of one or more skin penetration enhancers capable of percutaneous delivery of the macrolide related immunosuppressant through the skin. Conditions which can be treated employing the topical pharmaceutical compositions of the present invention include, but are not limited to, treatment of autoimmune related skin conditions, and the like and combinations thereof.

The following examples are provided to enable one skilled in the art to practice the invention and are merely illustrative of the invention. The examples should not be read as limiting the scope of the invention as defined in the claims.

### EXAMPLE

### Gel—Formula Composition

The ingredients used in this example are set forth below in Table 1.

TABLE 1

| | | | Range which |
|---|---|---|---|
| | | % w/w | can be used |
| Ingredients | Category | (in Example 1) | (% w/w) |
| Tacrolimus | API | about 0.10 | about 0.01- |
| (as Tacrolimus | | | about 5.00 |
| Monohydrate) | | | |
| N-Methyl-2- | Solubilizer & | about 5.00 | about 0.01- |
| Pyrrolidone | Penetration | | about 30.00 |
| (Pharmasolve ®) | Enhancer | | |
| Ethanol | Co-solvent for | about 25.00 | about 1.00- |
| | the solubilizer & | | about 90.00 |
| | Penetration enhancer | | |
| Propylene Glycol | Co-solvent for | about 10.00 | about 1.00- |
| | the solubilizer | | about 90.00 |
| Carbomer | Gel forming agent | about 0.40 | about 0.01- |
| (Carbopol 980) | | | about 10.00 |
| Triethanolamine | Neutralizing | about 0.60 | about 0.01- |
| | agent for the | | about 15.00 |
| | Gel forming agent | | |
| Purified Water | Aqueous Base | about 58.90 | about 0.10- |
| | | | about 99.90 |
| Total | | 100.00 | |

The composition of this example was prepared as follows:
1. Tacrolimus was dissolved in N-Methyl-Pyrrolidone to form a solution.
2. Ethanol and propylene glycol were added to the solution of step no. 1.
3. Carbomer was dispersed in purified water under high shear at high speed stirring to form a uniform fine dispersion.
4. The solution from step no. 2 was added to the dispersion of step no. 3 under high shear at high speed stirring.
5. Triethanolamine was added to the dispersion of step no. 4 under low shear at low speed stirring, to provide a clear gel formulation.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

1. A pharmaceutical gel composition comprising (a) a therapeutically effective amount of one or more active pharmaceutical ingredients comprising one or more macrolide related immunosuppressants or pharmaceutically acceptable salts or esters thereof; (b) one or more gel forming agents; and (c) an effective amount of one or more skin penetration enhancers capable of percutaneous delivery of the macrolide related immunosuppressant through the skin.

2. The pharmaceutical gel composition of claim 1, wherein the macrolide related immunosuppressant is selected from the group consisting of tacrolimus, pimecrolimus, sirolimus, acsomycin, everolimus, pharmaceutically acceptable salts or esters thereof and mixtures thereof.

3. The pharmaceutical gel composition of claim 1, wherein the skin penetration enhancer is selected from the group consisting of one or more volatile organic solvents, one or more nonvolatile organic solvents and mixtures thereof.

4. The pharmaceutical gel composition of claim 3, wherein the volatile organic solvents are selected from the group consisting of a C₁-C₂₀ aliphatic, cycloaliphatic and/or aromatic-aliphatic alcohols, each of which is monohydric or polyhydric, alcohol/water mixtures, saturated or unsaturated fatty alcohols having about 8 to about 18 carbon atoms, saturated or unsaturated fatty acids or esters thereof having about 8 to about 18 carbon atoms and mixtures thereof.

5. The pharmaceutical gel composition of claim 3, wherein the volatile organic solvent is a C₁-C₂₀ alcohol.

6. The pharmaceutical gel composition of claim 3, wherein the nonvolatile organic solvents are selected from the group consisting of pyrrolidones, polyol ethers, polyols and mixtures thereof.

7. The pharmaceutical gel composition of claim 6, wherein the pyrrolidones are selected from the group consisting of N-alkylpyrrolidones, vinyl pyrrolidone, 2-pyrrolidone, 2-pyrrolidone-5-carboxylic acid, N-(2-hydroxyethyl)-2-pyrrolidone or fatty acid esters thereof, 1-lauryl-4-methoxycarbonyl-2-pyrrolidone, N-tallowalkylpyrrolidones and mixtures thereof.

8. The pharmaceutical gel composition of claim of claim 1, wherein the skin penetration enhancer is diethylene glycol monoethyl ether.

9. The pharmaceutical gel composition of claim 6, wherein the polyol ethers are a C₂-C₃₀ polyol ether containing from 2 to about 10 hydroxyl groups.

10. The pharmaceutical gel composition of claim 6, wherein the polyol ethers are selected from the group consisting of ethylene glycol monomethyl ether, ethylene glycol monoethyl ether, ethylene glycol monobutyl ether, ethylene glycol monopropyl ether, ethylene glycol monophenyl ether, ethylene glycol monohexyl ether, diethylene glycol monoethyl ether, diethylene glycol monomethyl ether, triethylene glycol monomethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethyl glycol monohexyl ether, ethylene glycol phenyl ether and mixtures thereof.

11. The pharmaceutical gel composition of claim 6, wherein the polyols are selected from the group consisting of ethylene glycol, propylene glycol, butylene glycol, hexylene glycol, propylene glycol monocaprylate and mixtures thereof.

12. The pharmaceutical gel composition of claim 1, wherein the skin penetration enhancer comprises a pyrrolidone in at least one solvent selected from the group consisting of C₁-C₄ alcohols, C₁-C₄ alkylene glycols, C₁-C₄ polyalcohols, C₁-C₄ polyalkylene glycols and mixtures thereof.

13. The pharmaceutical gel composition of claim 1, comprising about 0.01% to about 5% w/w of the macrolide related immunosuppressant and about 0.01% to about 30% w/w of the skin penetration enhancer.

14. The pharmaceutical gel composition of claim 1, comprising about 0.02% to about 2% w/w of the macrolide related immunosuppressant and about 1% to about 10% w/w of the skin penetration enhancer.

15. The pharmaceutical gel composition of claim 1, wherein the macrolide related immunosuppressant is tacrolimus or a pharmaceutically acceptable salt or ester thereof and the skin penetration enhancer is a glycol ether.

16. The pharmaceutical gel composition of claim 1, comprising about 0.01% to about 5% w/w of tacrolimus or a pharmaceutically acceptable salt or ester thereof and about 0.01% to about 30% w/w of a glycol ether.

17. The pharmaceutical gel composition of claim 1, comprising about 0.02% to about 2% w/w of tacrolimus or a pharmaceutically acceptable salt or ester thereof and about 1% to about 10% w/w of a glycol ether.

18. The pharmaceutical gel composition of claim 1, further comprising one or more neutralizers.

19. The pharmaceutical gel composition of claim 18, wherein the pH of the composition is about 3.0 to about 6.5.

20. The pharmaceutical gel composition of claim 1, further comprising one or more pharmaceutically acceptable excipients.

21. The pharmaceutical gel composition of claim 1, further comprising one or more additional active pharmaceutical ingredients.

22. A process for preparing a pharmaceutical gel composition comprising:
(a) dissolving a therapeutically effective amount of one or more active pharmaceutical ingredients comprising one or more macrolide related immunosuppressants or pharmaceutically acceptable salts or esters thereof in one or more penetration enhancers to form a first solution;
(b) adding one or more solvents to the first solution;
(c) mixing one or more gel forming agents in water to form a second solution;
(d) mixing the second solution with the product of step (b).

23. The process of claim 22, wherein the macrolide related immunosuppressant is selected from the group consisting of tacrolimus, pimecrolimus, sirolimus, acsomycin, everolimus, pharmaceutically acceptable salts or esters thereof and mixtures thereof.

24. The process of claim 22, wherein the skin penetration enhancer is one or more volatile organic solvents, one or more nonvolatile organic solvents and mixtures thereof.

25. The process of claim 24, wherein the volatile organic solvents are selected from the group consisting of a C₁-C₂₀ aliphatic, cycloaliphatic and/or aromatic-aliphatic alcohols, each of which is monohydric or polyhydric, alcohol/water mixtures, saturated or unsaturated fatty alcohols having about 8 to about 18 carbon atoms, saturated or unsaturated fatty acids or esters thereof having about 8 to about 18 carbon atoms and mixtures thereof.

26. The process of claim 24, wherein the non-volatile organic solvents are selected from the group consisting of pyrrolidones, polyol ethers, polyols and mixtures thereof.

27. The process of claim 26, wherein the pyrrolidones are selected from the group consisting of N-alkylpyrrolidones, vinyl pyrrolidone, 2-pyrrolidone, 2-pyrrolidone-5-carboxylic acid, N-(2-hydroxyethyl)-2-pyrrolidone or fatty acid esters thereof, 1-lauryl-4-methoxycarbonyl-2-pyrrolidone, N-tallowalkylpyrrolidones and mixtures thereof.

28. The process of claim 26, wherein the polyol ethers are selected from the group consisting of ethylene glycol monomethyl ether, ethylene glycol monoethyl ether, ethylene glycol monobutyl ether, ethylene glycol monopropyl ether, ethylene glycol monophenyl ether, ethylene glycol monohexyl ether, diethylene glycol monoethyl ether, diethylene glycol monomethyl ether, triethylene glycol monomethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethyl glycol monohexyl ether, ethylene glycol phenyl ether and mixtures thereof.

29. The process of claim 26, wherein the polyols are selected from the group consisting of ethylene glycol, propylene glycol, butylene glycol, hexylene glycol, propylene glycol monocaprylate and mixtures thereof.

30. The process of claim 22, wherein the solvent is selected from the group consisting of C₁-C₄ alcohols, C₁-C₄ alkylene glycols, C₁-C₄ polyalcohols, C₁-C₄ polyalkylene glycols and mixtures thereof.

31. The process of claim 22, further comprising (e) mixing a neutralizing agent.

32. A method for delivering a macrolide related immunosuppressant or pharmaceutically acceptable salts or esters thereof through the skin in order to treat conditions situated on and beneath the skin comprising the step of topically administering an effective amount of a pharmaceutical gel composition comprising (a) a therapeutically effective amount of one or more active pharmaceutical ingredients comprising one or more macrolide related immunosuppressants or pharmaceutically acceptable salts or esters thereof; (b) one or more gel forming agents; and (c) an effective amount of one or more skin penetration enhancers capable of percutaneous delivery of the macrolide related immunosuppressant through the skin.

33. The method of claim 32, wherein the macrolide related immunosuppressant is selected from the group consisting of tacrolimus, pimecrolimus, sirolimus, acsomycin, everolimus, pharmaceutically acceptable salts or esters thereof and mixtures thereof.

34. The method of claim 32, wherein the pharmaceutical gel composition comprises about 0.01% to about 5% w/w of the macrolide related immunosuppressant or pharmaceutically acceptable salt or ester thereof and about 0.01% to about 30% w/w of the skin penetration enhancer.

35. The method of claim 32, wherein the pharmaceutical gel composition comprises about 0.02% to about 2% w/w of the macrolide related immunosuppressant or pharmaceutically acceptable salt or ester thereof and about 1% to about 10% w/w of the skin penetration enhancer.

36. The method of claim 32, wherein the macrolide related immunosuppressant is tacrolimus or a pharmaceutically acceptable salt or ester thereof and the skin penetration enhancer is a glycol ether.

37. The method of claim 32, wherein the pharmaceutical gel composition comprises about 0.01% to about 5% w/w of the macrolide related immunosuppressant or pharmaceutically acceptable salt or ester thereof and about 0.01% to about 30% w/w of a glycol ether.

38. The method of claim 32, wherein the pharmaceutical gel composition about 0.02% to about 2% w/w of the macrolide related immunosuppressant or pharmaceutically acceptable salt or ester thereof and about 1% to about 10% w/w of a glycol ether.
